(19) United States
(12) Patent Application Publication
(10) Pub. No.: US 2016/0143791 A1
(43) Pub. Date: May 26, 2016

(54) STRETCH RELEASE ARTICLES AND FASTENERS

(71) Applicant: 3M INNOVATIVE PROPERTIES COMPANY, St. Paul, MN (US)

(72) Inventors: Leigh E. Wood, Woodbury, MN (US); Steven J. Perron, St. Paul, MN (US)

(21) Appl. No.: 14/899,987

(22) PCT Filed: Jun. 13, 2014
(86) PCT No.: PCT/US2014/042315

§ 371 Date: Dec. 18, 2015

Related U.S. Application Data
(60) Provisional application No. 61/837,418, filed on Jun. 20, 2013.

(51) Int. Cl. A61F 13/56; A61L 15/58; A44B 18/00

(52) U.S. Cl. CPC A61F 13/5616; A44B 18/008; A61L 15/58

(57) ABSTRACT

Stretch release articles and fasteners comprising an elastic backing having pressure sensitive adhesive on one side of the elastic backing and bonds or bond elements not formed from pressure sensitive adhesive on the other side of the elastic backing. The pattern of pressure sensitive adhesive on the one side and the pattern of bonds or bonding elements on the other side do not substantially overlap when projected onto a common reference plane that is coplanar with the elastic backing. The stretch release articles and fasteners can be used in a variety of applications, including medical, industrial and consumer products.

### FIELD OF INVENTION

Stretch release articles and fasteners. The articles and fasteners may be applied to a substrate and subsequently stretch released without damage to the substrate. The stretch release articles and fasteners can be used in a variety of applications, including medical, industrial and consumer products.

### BACKGROUND

Stretch release fasteners provide temporary bonding solutions in a wide variety of commercial and industrial applications. These fasteners can be easily removed from a bonded substrate by stretching the fastener lengthwise in a direction substantially parallel to the plane of the substrate. Because the adhesion substantially degrades as the adhesive is elongated, the stretching action enables the adhesive to be conveniently detached without damaging the underlying substrate. These fasteners are commonly used to bond two different substrates to each other, thus allowing two adhesively bonded materials to be separated from each other without damage to either substrate.

Commercial stretch release fasteners include COMMAND brand adhesive tapes sold by 3M Company, St. Paul, Minn. and POWER-STRIPS brand self-adhesive tapes sold by Beiersdorf AG, Hamburg, Germany. These products can be optionally provided with long discrete strips of a pressure sensitive adhesive with a pull tab at one end to facilitate stretching of the strips during removal. Optionally, the adhesive areas can be protected using a release liner prior to use. In some cases, it is advantageous for the tape to undergo inelastic deformation as it stretches to avoid sudden recoil, or “snap,” when the adhesive fully detaches. An ancillary benefit of inelastic deformation during stretching is that such products do not return to their original shape, thereby indicating if the product has been previously used or tampered with.

Stretch release fasteners can be especially useful in bonding to soft and/or delicate surfaces. For example, such fasteners have been used for temporary attachment of paper articles, such as for holding posters, signs, or other large format graphics, or even bonding to skin in medical tape, wound or surgical dressing, athletic tape, surgical drape, and medical device applications. Finally, these fasteners can also be used to facilitate attachment of other articles to clothing, as commonly used in adult incontinence pads or feminine hygiene products such as absorbent sanitary napkins, which are adhered to undergarments. These types of applications pose unique challenges not only because of the flexibility of the substrate but also the need to reconcile the demand for high levels of adhesion with the need to avoid inadvertent damage to the substrate during removal.

### SUMMARY

Engineering a releasable adhesive fastener can present unique challenges with respect to user experience. In some applications, users find that a peeling action is a more intuitive way to remove an article adhesively bonded to a substrate. Yet, detachment from the substrate does not effectively occur with conventional stretch release fasteners unless the adhesive is stretched lengthwise in a direction substantially parallel to the plane of the substrate surface. Peeling conventional stretch release fasteners from a substrate may require undue force or, even worse, leave adhesive residue on the surface of the substrate. While these problems may be addressed by using a less aggressive adhesive, such a solution is often inadequate when bonding to garments.

Articles intended for adhesion to garments can face particular challenges resulting from certain types of garment movements during use. These movements may include longitudinal and transverse extension as well as twisting movements, which may impose stresses on the adhesive. A common failure mode for feminine hygiene articles is the partial detachment or shifting of the adhesive induced by twisting and shearing type movements of the garment relative to feminine hygiene articles. These movements, which often occur as a result of walking, can cause the hygiene article to slowly migrate from its intended position. If the stresses are sufficiently large, these movements can even cause the adhesive to completely detach from the undergarment. Also problematic is “bunching” of the garment, which can cause a partially detached adhesive to buckle and become permanently adhered to itself.

The articles and fasteners of the present disclosure can be joined to a substrate and subsequently stretch released in a direction parallel to the substrate or in a direction at an angle to the substrate (e.g., peal action) without leaving significant adhesive residue on the substrate. Moreover, the articles and fasteners of the present disclosure are less prone to detach from a substrate during, for example, any twisting and/or shearing motion of the substrate during use.

These problems have been overcome by creating stretch release articles and fasteners comprising an elastic backing having pressure sensitive adhesive on one side of the elastic backing and bonds or bond elements not formed from pressure sensitive adhesive on the other side of the elastic backing. The pattern of pressure sensitive adhesive on the one side and the pattern of bonds or bond elements on the other side do not substantially overlap when projected onto a common reference plane that is coplanar with the elastic backing. This configuration results in a stretch release article or fastener that provides a reliable bond to substrates yet can be removed relatively cleanly and easily from the substrates, including delicate substrates such as paper or undergarments that may otherwise be damaged or destroyed when using conventional adhesive articles or fasteners. As a further benefit, these articles and fasteners have been found to perform well when subject to twisting and shearing type motions.

In one embodiment, the invention provides a stretch release article comprising: an elastic backing having a first side and a second side opposite the first side; a pressure sensitive adhesive on the first side of the elastic backing, the pressure sensitive adhesive exhibiting a first pattern; and a component joined to the second side of the elastic backing by bonds exhibiting a second pattern; where the bonds are not created by a pressure sensitive adhesive, and where the first and second patterns do not substantially overlap with each other when projected in the same plane.

In another embodiment, the invention provides a stretch release fastener comprising: an elastic backing having a first side and a second side opposite the first side; a pressure sensitive adhesive on the first side of the elastic backing, the pressure sensitive adhesive exhibiting a first pattern; a bonding element on the second side of the elastic backing, the bonding element exhibiting a second pattern; where the bonding element is not a pressure sensitive adhesive, and where the first and second patterns do not substantially overlap with each other when projected in the same plane.

As used herein, the terms “including,” “comprising,” or “having” and variations thereof encompass the items listed thereafter and equivalents thereof, as well as additional items. All numerical ranges are inclusive of their endpoints and non-integral values between the endpoints unless otherwise stated. Terms such “top,” “bottom,” and the like are only used to describe elements as they relate to one another, but are in no way meant to recite specific orientations of an article or apparatus, to indicate or imply necessary or required orientations of an article or apparatus, or to specify how an article or apparatus described herein will be used, mounted, displayed, or positioned in use.

The above summary of the present disclosure is not intended to describe each disclosed embodiment or every implementation of the present disclosure. The description that follows more particularly exemplifies illustrative embodiments. It is to be understood, therefore, that the drawings and following description are for illustration purposes only and should not be read in a manner that would unduly limit the scope of this disclosure.

### BRIEF DESCRIPTION OF THE DRAWINGS

FIG. 1A is a cross-sectional view of a stretch release article;

FIG. 1B is a plan view showing a first pattern of pressure sensitive adhesive and a second pattern of bonds in the stretch release article of FIG. 1A;

FIG. 2A is a plan view showing a suitable combination of first and second patterns;

FIG. 2B is a cross-sectional view of FIG. 2A taken along 2B-2B.

FIG. 2C is a cross-sectional view of FIG. 2A taken along 2C-2C.

FIG. 3A is an plan view showing another suitable combination of first and second patterns;

FIG. 3B is a cross-sectional view of FIG. 3A taken along 3B-3B;

FIG. 3C is a cross-sectional view of FIG. 3A taken along 3C-3C;

FIG. 3D is a cross-sectional view of FIG. 3A taken along 3D-3D;

FIG. 4A is a plan view showing yet another suitable combination of first and second patterns;

FIG. 4B is a cross-sectional view of FIG. 4A taken along 4B-4B;

FIG. 4C is a cross-sectional view of FIG. 4A taken along 4C-4C;

FIG. 4D is a cross-sectional view of FIG. 4A taken along 4D-4D;

FIG. 5A is a top plan view of a stretch release article;

FIG. 5B is a bottom plan view of the stretch release article in FIG. 5A;

FIGS. 5C-5E are fragmentary bottom plan views of FIG. 5B showing three different combinations of first and second patterns;

FIG. 6 is an elevated side view of the stretch release article in FIG. 1A as it is peeled away from a substrate;

FIG. 7A is a plan view of the stretch release article in Example 1;

FIG. 7B is a plan view of the stretch release article in Example 3;

FIG. 8 is a cross-sectional view of a dual-sided fastener; and

FIG. 9 is a cross-sectional view of another dual-sided fastener.

### DETAILED DESCRIPTION

Described in further detail herein are articles and fasteners that use a stretch release adhesive along with associated methods of making and using the same. Stretch release articles and fasteners can be customized for any of a number of applications including medical, industrial, and consumer products. In particular, these constructions can be specialized for bonding articles to various substrates, including both rigid substrates (e.g., walls) and flexible substrates (e.g., clothing).

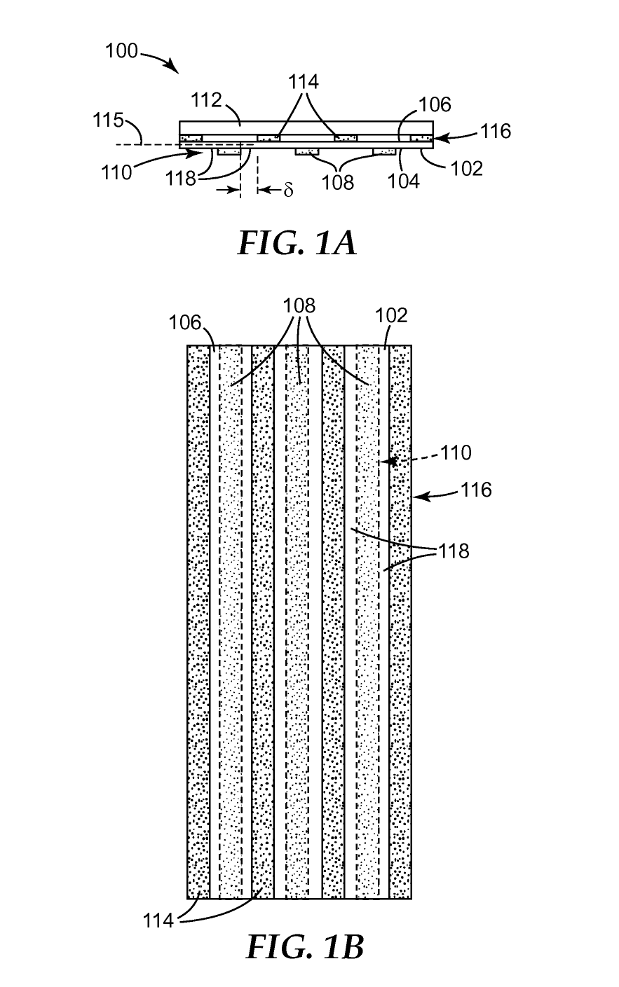

An exemplary stretch release article of the present disclosure is illustrated in FIG. 1A. The stretch release article 100 comprises an elastic backing 102 having a first side 104 and a second side 106 opposite the first side 104. A pressure sensitive adhesive 108 is located on the first side 104 of the elastic backing 102 and exhibits a first pattern 110. The pressure sensitive adhesive 108 is used to releasably join the stretch release article 100 to a substrate (not shown). A release liner (also not shown) may cover the pressure sensitive adhesive and be removed prior to application of the stretch release article 100 to the substrate. Any conventional release liner may be used. Exemplary release liners include olefins (e.g., polyethylene and polypropylene) and coated papers (e.g., silicone coated papers).

A component 112 is joined to the second side 106 of the elastic backing 102 by bonds 114 exhibiting a second pattern 116. The component 112 can be any functional and/or ornamental element capable of being joined to a substrate through the pressure sensitive adhesive 108 on the first side 104 of the elastic backing 102. Exemplary components 112 include feminine hygiene pads, wound dressings, pet pads, wall fixtures, and signage. In FIG. 1A, the component 112 and elastic backing 102 are the same size and rectangular in shape. However, it should be understood that the component 112 and elastic backing 102 can have a variety of shapes and that the component 112 may or may not be the same size and/or shape as the elastic backing 102.

The bonds 114 that join the component 112 to the elastic backing 102 may be created by a variety of techniques, including gluing, welding (e.g., thermal, chemical and ultrasonic), stapling, sewing, and fastening with a hook-and-loop mechanical fastener, but the bonds 114 are typically not created by a pressure sensitive adhesive. The choice of method will depend to some extent on the nature of the materials making up the elastic backing and component of a particular article. However, bonding methods are well-known to those skilled in the art, and materials and equipment used to create such bonds are commercially available.

The first pattern 110 of pressure sensitive adhesive 108 and second pattern 116 of bonds 114 are more clearly illustrated in FIG. 1B. FIG. 1B is a plan view of the stretch release article 100 in FIG. 1A where the component 112 has been removed. The elastic backing 102 is translucent so that both patterns 110, 116 are visible, but the elastic backing need not be translucent generally. The term “pattern”, as used herein, refers to a two-dimensional configuration of pressure sensitive adhesive, bonds or bond elements (introduced below) on the elastic backing 102. In preferred embodiments, each pattern has a defined repeat unit that is replicated across the elastic backing. The repeat units are further characterized by a repeat dimension, corresponding to the shortest distance a repeat unit is moved to superimpose on an adjacent repeat unit of the pattern. More than one repeat dimension may exist—for example, two different repeat dimensions may be oriented along respective x and y axes of the two-dimensional pattern.

Referring back to FIG. 1B, the pressure sensitive adhesive 108 is arranged in a first pattern 110 of parallel strips that extend across the first side 104 of the elastic backing 102. The bonds 114 are similarly arranged in a second pattern 116 of parallel strips that extend across the second side 106 of the elastic backing 102. Although this particular embodiment shows three pressure sensitive adhesive strips on the first side 104 and four bond strips on the second side 106 of the elastic backing 102, it should be understood that more or fewer strips may be used on the first and second sides 104, 106. Additionally, although all strips extend from one end of elastic backing 102 to the other, it should be understood that one or more strips on the first and second sides 104, 106 may terminate a distance from one or both ends of the elastic backing 102. For example, in some embodiments, the strips of pressure sensitive adhesive do not extend all the way to the ends of the elastic backing. The pressure sensitive adhesive free ends can function as a finger tab to assist with removing the article from the substrate.

As illustrated in FIGS. 1A and 1B, the first pattern 110 of pressure sensitive adhesive 108 does not overlap with the second pattern 116 of bonds 114 when the first and second patterns 110,116 are projected onto a common reference plane 115 that is coplanar with the elastic backing 102 (i.e. projected in the same plane). As a result, areas of elastic backing 102 that contain pressure sensitive adhesive 108 on the first side 104 do not contain bonds 114 on the second side 106 and vice versa. Stated inversely, the configuration avoids having regions of the elastic backing 102 where pressure sensitive adhesive 108 is directly below bonds 114.

The first and second patterns 110, 116 as projected onto the reference plane 115 are offset from each other by a transverse gap 118. As shown, the gap 118 is characterized by a gap width 6, and extends in a direction perpendicular to the strips along the plane of the elastic backing 102. Areas of the elastic backing 102 within the gap 118 are bare; that is, these areas do not contain pressure sensitive adhesive or bonds on either the first or second sides 104,106, respectively, of the elastic backing 102.

Although the gap width δ is constant in the embodiment depicted in FIGS. 1A and 1B, it could be variable for other embodiments. For example, if the pressure sensitive adhesive strips and bond strips in FIGS. 1A and 1B were not parallel (e.g., strips have wavy rather than linear edges), the gap width δ would have a range of values. When the gap width δ is not constant, the gap is characterized by an average gap width δ based on an integrated average of differential gap widths along the perimeter of each pattern element.

In some embodiments, the gap has a gap width δ (or average gap width δ) of at least about 10 percent, at least about 25 percent, at least about 50 percent, at least about 100 percent, or at least about 200 percent of the repeat dimension for either the first or second pattern. In some embodiments, the gap width δ (or average gap width δ) is at most about 500 percent, at most about 400 percent, at most about 300 percent, at most about 250 percent, or at most about 200 percent of the repeat dimension for either the first or second pattern. In some embodiments, the gap width δ (or average gap width δ) is about 10 percent to about 500 percent of the repeat dimension for either the first or second pattern.

The precise nature of the first and second patterns need not be limiting. While the first and second patterns illustrated in FIGS. 1A and 1B define a repeat pattern of elongated strips, many other patterns are possible. Each pattern can include a repeating pattern of elements or a random pattern of elements. Those elements may include, for example, a pattern of strips, triangles, rectangles, circles, any number of other two-dimensional shapes, or combinations thereof. Further, a pattern can include continuous features, discontinuous features, or combination thereof. For example, a suitable pattern could include one or more continuous spirals. Elements within a pattern can also be more than one size. For example, the pattern may consist of circles having a variety of diameters.

As illustrated in FIGS. 1A and 1B, the first and second patterns do not overlap (i.e., the gap width δ is greater than zero). However, for the purposes of this disclosure, it is only necessary that the first and second patterns do not substantially overlap with each other. The first and second patterns do not substantially overlap when the area of overlap is about 20% or less of the area occupied by the first and second patterns when projected onto a common reference plane. For example, assume the first pattern and second pattern comprise vertical strips that are 6 mm wide by 30 mm long, and the strip in the first pattern overlaps the strip in the second pattern by 1 mm. The area of overlap is 30 mm² (i.e., 1 mm×30 mm), the area occupied by the first and second patterns when projected into a common reference plane is 330 mm² (i.e., 11 mm×30 mm), and the percentage of overlap is 9% (i.e, [30 mm²/330 mm²]×100%). Preferably, the area of overlap is less than about 15%, less than about 10%, less than about 5%, or 0% of the area occupied by the first and second patterns when projected onto a common reference plane. In embodiments where the first and second patterns are overlapping parallel strips, the gap width δ will have a negative value in regions of overlap. In embodiments where the first and second patterns are parallel strips where the edge of an element from the first pattern is in perfect registration with the edge of an element from the second pattern, the gap width δ will be zero.

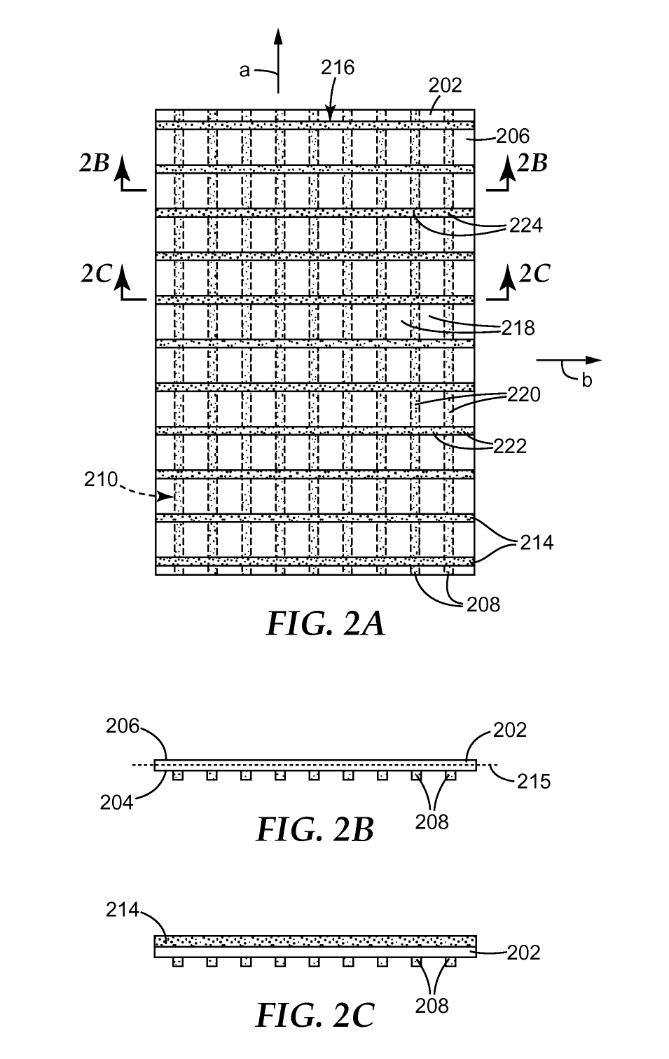

Suitable alternative combinations of first and second patterns are illustrated in FIGS. 2-4. The combination of patterns share many of the same elements and features described above with respect to the stretch release article in FIGS. 1A and 1B. Accordingly, elements and features corresponding to elements and features in the illustrated embodiment of FIGS. 1A and 1B are provided with the same reference numerals in the 200, 300 and 400 series. Reference is made to the description above accompanying FIGS. 1A and 1B for a more complete description of the features and elements (and alternatives to such features and elements) of the embodiments illustrated in FIGS. 2-4.

Referring to FIGS. 2A-2C, the first pattern 210 of pressure sensitive adhesive 208 consists of a set of elongated strips extending in a first direction “a” and the second pattern 216 of bonds 214 consists of a set of elongated strips extending in a second direction “b” where the second direction “b” is perpendicular to the first direction “a”. In this embodiment, the first and second patterns 210, 216 overlap each other at intersection points when the respective patterns 210, 216 are projected onto a common reference plane 215 that is coplanar with the elastic backing 202.

FIG. 2A highlights four distinct regions 218, 220, 222, 224 that can be identified with respect to either the first or second side 204, 206 of the elastic backing 202. These include: (i) a periodic two-dimensional array of gap regions 218 where there is no pressure sensitive adhesive 208 or bonds 214 present on either side of the elastic backing 202, (ii) coated regions 220 containing only pressure sensitive adhesive 208, (iii) bonded regions 222 containing only bonds 214, and (iv) coated-bonded regions 224 containing pressure sensitive adhesive 208 on the first side 204 of the elastic backing 202 and bonds 214 on the second side 206 of the elastic backing 202.

The coated-bonded regions 224 coincide with areas of overlap between the first and second patterns 210, 216 when projected onto a common reference plane 215 that is coplanar with the elastic backing 202. The presence of the coated-bonded regions 224 can be significant during stretch removal because the elastic backing 202 is constrained on both sides 204, 206, preventing the pressure sensitive adhesive 208 from stretching to reduce the force needed to break the bond to the substrate. Having limited localized regions where stretch removal is constrained allows a substrate and stretch release article to be effectively “pinned” to each other in some applications. These pinned regions act to retard adhesive detachment along the coated-bonded regions 224 and may be desirable in certain applications.

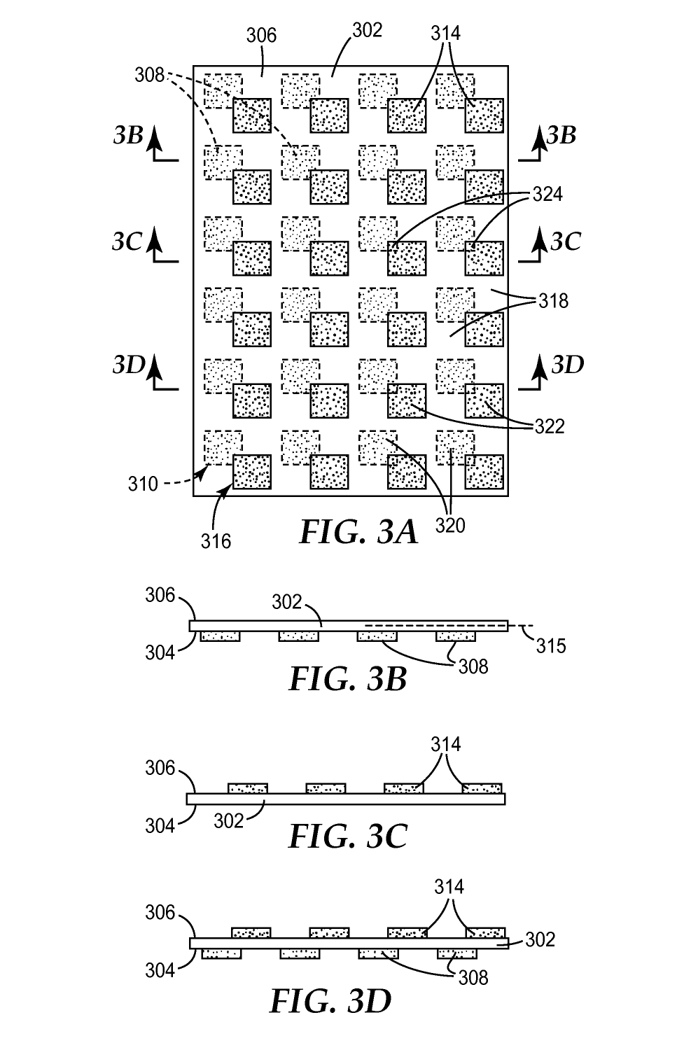

Another exemplary embodiment displaying a limited degree of overlap between the first pattern 310 of pressure sensitive adhesive 308 and second pattern 316 of bonds 314 is shown in FIGS. 3A-3D. The first pattern 310 of pressure sensitive adhesive 308 is a regular array of discrete rectangles extending across the first side 304 of the elastic backing 302. The second pattern 316 of bonds 314 is a regular array of discrete rectangles extending across the second side 306 of the elastic backing 302, where the second pattern 316 is diagonally offset from the first pattern 310. The first and second patterns 310, 316 slightly overlap each other when the respective patterns 310, 316 are projected onto a common reference plane 315 that is coplanar with the elastic backing 302. Similar to the configuration in FIGS. 2A-2C, the configuration in FIGS. 3A-3D includes gap regions 318, coated and bonded regions 320, 322, respectively, and coated-bonded regions 324.

Related embodiments include articles having inverse “checkboard”-type patterns that do not substantially overlap with each other. As another possibility, a gap could be present between checkboard patterns where the corners of the opposing patterns are diagonally spaced apart from each other.

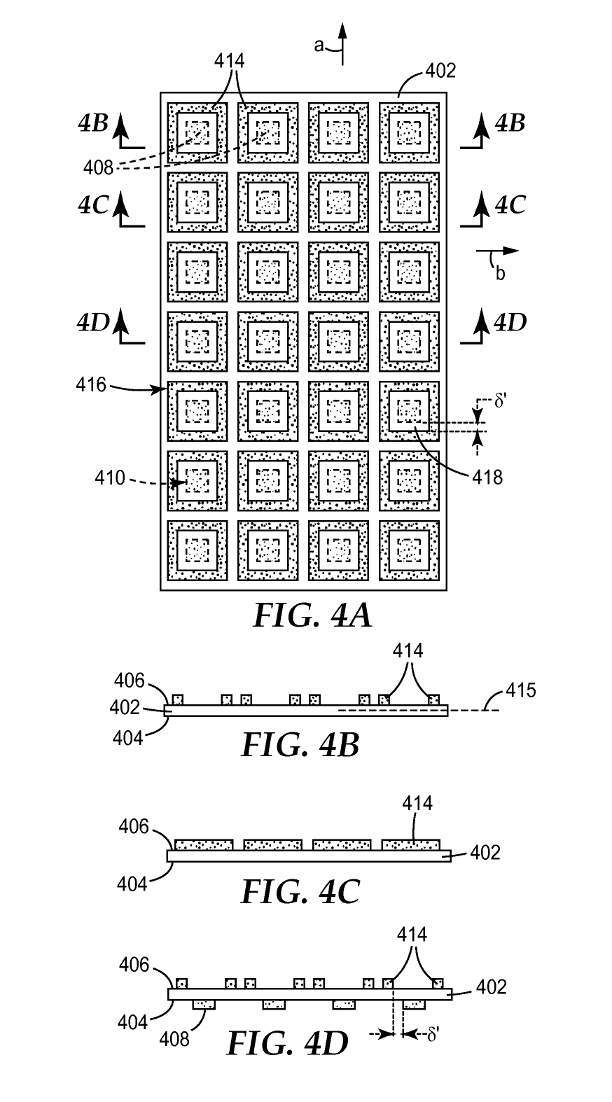

Yet another exemplary configuration of first and second patterns is illustrated by FIGS. 4A-4D. The first pattern 410 of pressure sensitive adhesive 408 is a regular array of closed squares. The second pattern 416 of bonds 414 is a regular array of open squares that are concentric with those in the first pattern 410. The closed squares in the first pattern 410 lie within but do not touch the open square of the second pattern 416 when the respective patterns 410, 416 are projected onto a common reference plane 415 that is coplanar with the elastic backing 402. The first and second patterns 410, 416 are separated by a gap 418 having a fixed, pre-defined gap width δ′. As shown, the gap width δ′ is the same along both vertical “a” and horizontal “b” directions. The gap width δ′ is largely analogous to the gap width δ defined in FIG. 1A.

The squares of the first pattern 410 need not be closed. In an alternative embodiment, the squares of the first pattern are “open,” but sufficiently small in size relative to the squares in the second pattern to satisfy the condition that there is no substantial overlap between the first and second patterns.

Due to the four-fold symmetry of the first and second patterns 410, 416 in FIG. 4A, the elastic backing 402 would exhibit the same stretch characteristics along the vertical “a” direction and horizontal “b” direction. By using rectangles instead of squares, it is possible to introduce asymmetric stretching characteristics such that the stretch release article more easily releases in one direction than another.

The above combinations of first and second patterns are an illustrative but not exhaustive list of combinations. As previously mentioned, it is only necessary that the first and second patterns do not substantially overlap.

In some embodiments, the first and second patterns collectively extend across an area representing at least about 10 percent, at least about 15 percent, or at least about 25 percent of the overall surface area of the elastic backing (the combined surface area of the first and second sides). In some embodiments, the first and second patterns collectively extend across an area representing at most about 60 percent, at most about 30 percent, or at most about 10 percent of the overall surface area of the elastic backing. In some embodiments, the first and second patterns collectively extend across an area representing about 10 percent to about 60 percent of the overall surface area of the elastic backing.

In some embodiments, the first pattern of the pressure sensitive adhesive and the second pattern of bonds each cover the same percentage of area on the first and second sides of the elastic backing, respectively. In other embodiments, the first pattern of pressure sensitive adhesive covers a larger area of the elastic backing than the second pattern of bonds. In yet other embodiments, the first pattern of pressure sensitive adhesive covers a smaller area of the elastic backing than the second pattern of bonds.

The coating weight of the pressure sensitive adhesive, which relates to adhesive layer thickness, can be adjusted as needed based on the materials and application. In some embodiments, the pressure sensitive adhesive has a coating weight of at least about 5 gsm, at least about 10 gsm, at least about 20 gsm, at least about 35 gsm, or at least about 50 gsm of the first side of the elastic backing. In some embodiments, the pressure sensitive adhesive has a coating weight of at most about 100 gsm, at most about 90 gsm, at most about 75 gsm, at most about 60 gsm, or at most about 50 gsm of the first side of the elastic backing. In some embodiments, the pressure sensitive adhesive has a coating weight of about 5 gsm to about 100 gsm of the first side of the elastic backing.

In some cases, it is desirable for the pressure sensitive adhesive to have sufficient elasticity so not to interfere with the recovery of the elastic backing after it has been stretched during peel. This can be especially beneficial, for example, in a consumer application where the stretch release article may require re-positioning on a garment after being improperly positioned on the first attempt. In other cases, it may be desirable for the pressure sensitive adhesive to prevent the elastic backing from fully recovering after use. This would be particularly beneficial where the article or fastener is intended for single use and it is desirable to know if the article or fastener was previously used or tampered with.

Exemplary materials that may be used to construct the stretch release articles and fasteners of the present invention are provided below.

### Elastic Backing

Suitable materials for the elastic backing include polymeric sheet materials that display high elasticity, high strength, and high tensile strength to function properly during removal of the stretch release article. Exemplary elastic backings preferably have an elastic recovery of at least about 70 percent, at least about 80 percent, or at least about 90 percent based on an elongation of 100 percent.
Percent elongation=[(Lₛ−Lₒ)/Lₒ]×100, where Lₒ is original length and Lₛ is stretched length.
Percent recovery (after a defined period of time)=[(Lₛ−Lᵣ)/Lₒ]×100, where Lₒ and L₅ are as defined above and Lᵣ is length after relaxation from stretch.

It is also preferred that the elastic backing have a sufficient range of elongation that enables removal of the adhesive from the underlying substrate on demand. In some embodiments, the elastic backing has a lengthwise elongation at break of at least about 50 percent, at least about 150 percent, or at least about 350 percent. In some embodiments, the lengthwise elongation at break is at most about 1,200 percent, at most about 900 percent, or at most about 700 percent. In some embodiments, the lengthwise elongation at break is about 50 to about 1,200 percent.

Examples of materials particularly suitable for the elastic backing of the stretch release article include any material capable of being formed into a thin layer and exhibiting elastomeric properties at ambient conditions. Elastomeric means that the material will substantially resume its original shape after being stretched. The elastomer can be both pure elastomers and blends with an elastomeric phase or content that will still exhibit substantial elastomeric properties at room temperature.

Suitable elastomers include such elastomeric polymers known to those skilled in the art as AB and ABA block copolymers. Star or radial block copolymers may also be suitable in the present invention. Elastomeric block copolymers are typically thermoplastic rubbers that have a rubbery midblock with at least two high glass transition temperature endblocks. Block copolymers are described, for example, in U.S. Pat. No. 3,562,356 (Nyberg et al.); U.S. Pat. No. 3,700,633 (Wald et al.); U.S. Pat. No. 4,116,917 (Eckert); and U.S. Pat. No. 4,156,673 (Eckert).

There are at least two basic classes of these polymers. In one class, the midblock is an unsaturated rubber. Examples of two types of polymers in this class are known to those skilled in the art as styrene-butadiene-styrene (SBS) and styrene-isoprene-styrene (SIS) block copolymers. In the other class, the midblock is a saturated olefin rubber. Examples of two types of polymers in this class are known to those skilled in the art as styrene-ethylene-butadiene-styrene (SEBS) and styrene-ethylene-propylene-styrene (SEPS) block copolymers. SIS, SBS, SEBS, and SEPS block copolymers are useful in the present invention, with SIS block copolymers being preferred because of the elastomeric properties exhibited by these polymers.

Other useful elastomeric compositions may include elastomeric polyurethanes, ethylene copolymers such as ethylene vinyl acetates, ethylene/propylene copolymer elastomers or ethylene/propylene/diene terpolymer elastomers. Blends of these elastomers with each other or with modifying non-elastomers may also be useful. For example, up to 50 weight percent, but preferably less than 30 weight percent, of polymers may be added such as poly(alpha-methyl)styrene, polyesters, epoxies, polyolefins, e.g., polyethylene or certain ethylene/vinyl acetates, preferably those of higher molecular weight, or coumarone-indene resin.

The elastic backing preferably has a composition that is compatible with the adhesive or adhesives used in the construction. For example, a pressure sensitive adhesive disposed thereon should form a strong bond with the surface of the elastic backing, thereby avoiding undesirable adhesive transfer during removal of the stretch release article from a substrate. Another consideration is the problem of cross-contamination which could occur when the elastic backing is placed in contact with certain adhesives. In some preferred constructions, the elastic backing includes a sandwich construction where a protective polyolefin skin layer is disposed on each major surface of an elastomeric core layer. Protective layers can prevent tackifiers in an adhesive from migrating into the elastic film and causing the adhesive to lose tack over time.

Other aspects of elastomeric materials suitable for the elastic backing are described in U.S. Pat. No. 5,376,430 (Swenson et al.); U.S. Pat. No. 5,691,034 (Krueger et al.); and U.S. Pat. No. 6,436,529 (Deeb et al.).

The elastic backing can be made by any method of film forming presently known in the art, such as extrusion, co-extrusion, solvent casting, foaming, and the like. Use of a non-woven web to form the elastic backing is also possible. Such technology is described, for example, in U.S. Publication Nos. 2012/0329351 (Mehta, et al.); US 2011/0256791 (Seidel, et al.); US 2005/0186879 (Martin); and U.S. Pat. No. 7,659,218 (Nishiguchi, et al.); U.S. Pat. No. 7,405,171 (Tsujiyama, et al.); U.S. Pat. No. 5,238,733 (Joseph, et al.); U.S. Pat. No. 5,997,989 (Gessner, et al.).

The elastic backing can have any thickness so long as it possesses sufficient integrity to be easily processed and handled. Preferably, the elastic backing has a thickness ranging from about 10 micrometers to 250 micrometers. In the preferred range, thinner backings can sometimes provide easier removal than thicker ones.

### Pressure Sensitive Adhesive

A pressure sensitive adhesive is used to join the stretch release article (or fastener) to a substrate. The particular adhesion properties can be at least partially dependent on the mode of measurement. Preferred adhesion properties generally range from about 13 N/dm to about 200 N/dm, preferably from about 25 N/dm to about 100 N/dm, at a peel angle of 180 degrees, measured according to PSTC-1 and PSTC-3 and ASTM D 903-83 at a peel rate of 12.7 cm/min. An elastic backing having a higher tensile strength may be used for adhesives with high peel adhesion.

Pressure sensitive adhesives suitable for this invention include tackified rubber adhesives, such as natural rubber, olefins, silicones, polyisoprene, polybutadiene, polyurethanes, SIS and SBS block copolymers, and other elastomers, and tackified or untackified acrylic adhesives such as copolymers of isooctylacrylate and acrylic acid, which can be polymerized by radiation, solution, suspension, or emulsion techniques.

For applications such as feminine hygiene pads, it may be preferred to use tackified synthetic rubber type pressure sensitive adhesives rather than radiation or otherwise cross-linked pressure sensitive adhesives. Advantageously, the former pressure sensitive adhesives can be melted in the bulk and subsequently applied to the backing. These materials include, for example, tackified SIS block copolymer adhesives.

The pressure sensitive adhesive can be applied to the elastic backing by a variety of known methods. For example, the pressure sensitive adhesive can either be directly coated onto the elastic backing, or formed as a separate layer and then later laminated to the elastic backing. As another example, the pressure sensitive adhesive could be coated onto a substrate and then adhesively coupled to the elastic backing. In one exemplary embodiment, the elastic backing is part of a continuously conveyed web and pressure sensitive adhesive is pattern coated onto the elastic backing using intermittent adhesive applicators.

To improve adhesion of the pressure sensitive adhesive, the elastic backing can optionally be pretreated prior to the coating or the laminating step by corona discharge, plasma discharge, flame treatment, electron beam irradiation, ultraviolet radiation, and/or chemical priming.

### Component

The component is that portion of the stretch release article that is bonded to the side of the elastic backing opposite the pressure sensitive adhesive. The component can be an ornamental and/or functional element. Exemplary components include disposable hygiene articles (e.g., feminine hygiene pads and adult incontinence pads), wound dressings, bed pads, pet pads, outerwear, undergarments, carpeting, light fixtures, pictures, signs, name tags, and poster board. The component may be complete or incomplete. For example, the component may be a picture frame into which a consumer will insert a personalized picture. Or, the component may be a poster board to which a consumer will add information for display.

The component is attached to a substrate through the pressure sensitive adhesive side of the elastic backing. The substrate can be just about anything to which the pressure sensitive adhesive will adhere. Exemplary substances include walls, ceilings, floors, skin, sheets, outwear and undergarments. In some instances, the component and substrate are interchangeable. For example, the elastic backing can be bonded to a feminine hygiene pad and the feminine hygiene pad releasably joined to the undergarment through the pressure sensitive adhesive side of the elastic backing. In an alternative configuration, the elastic backing can be bonded to an undergarment and an undergarment releasably joined to a feminine hygiene pad through the pressures sensitive adhesive side of the elastic backing. In the former case, the stretch release article includes the feminine hygiene pad (i.e., the substrate is the undergarment); in the latter case, the stretch release article includes the undergarment (i.e., the substrate is the feminine hygiene pad).

In some embodiments, the component is permanently bonded (e.g., by hot melt adhesive) to the elastic backing such that removal from the elastic backing would at least partially destroy the elastic backing and/or component. In some embodiments, the component is releasably bonded (e.g., by hook-and-loop mechanical fastener) to the elastic backing such that removal would leave the component and elastic backing relatively intact.

### General Method of Use

The stretch release articles of the present disclosure are releasably joined to a substrate through the pressure sensitive adhesive side of the elastic backing. Due to the elastic properties of the backing and the lack of substantial overlap between the pressure sensitive adhesive on one side and bonds on the other side of the elastic backing, the attachment between the article and substrate is less affected by twisting and/or sheering motions during use. The elastic backing is able to absorb the motion and reduce the likelihood that the article will separate from the substrate. At the same time, when desired, the article can be cleanly separated from the substrate by either a peel or sheering motion.

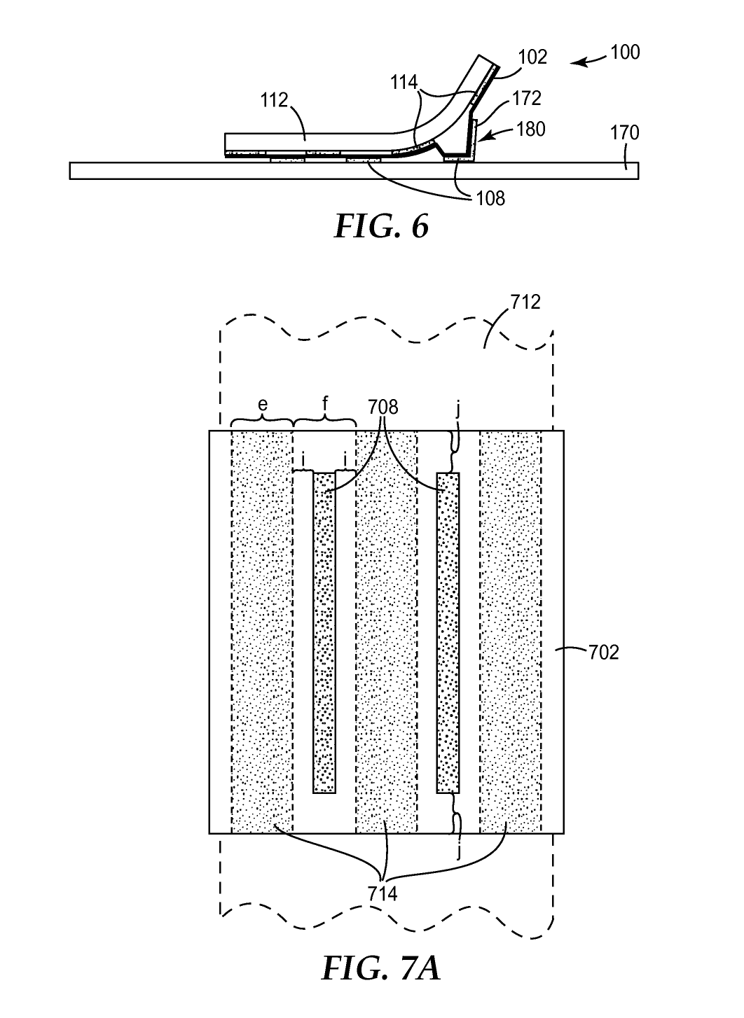

FIG. 6 illustrates an exemplary mode of detaching the stretch release article 100 in FIG. 1 from a substrate 170. The user grasps the article 100 on one side and peels it in a direction substantially perpendicular to the longitudinal axis of the strips of pressure sensitive adhesive 108. As a result of this peeling motion, the component 112 and elastic backing 102 remain bonded to each other while the elastic backing 102 and first strip 172 of pressure sensitive adhesive 108 collectively stretch. This stretching takes place along an area 180 of the article 100 that is devoid of bonds 114 and causes the pressure sensitive adhesive 108 to cleanly detach (i.e., release) from the substrate 170. Advantageously, the detachment operates in peel mode and therefore does not require stretching of the elastic backing 102 parallel to the plane of the substrate 170 as with conventional stretch release products.

As a further advantage, a wide range of peel angles can be used to detach the article 100 from the substrate 170. For example, if the component is relatively flexible, the elastic backing 102 can be stretched by pulling the elastic backing 102 away from the substrate 170 at a peal angle of at least 90 degrees, at least 120 degrees, or even at least 135 degrees relative to the substrate 170. If the component is relatively rigid, the peel angles are typically much less.

Although not shown here, the elastic backing 102 could include a non-adhesive pull tab that functions as a finger tab to peel the elastic backing from the substrate 170.

Detachment of the stretch release articles can also be actuated along different directions and provide different results. For example, the stretch release article 100 can be peeled away from the substrate 170 along a direction oriented 90 degrees from the peel direction illustrated in FIG. 6. This peel mode would be achieved, for example, by removing the article from a substrate in a direction parallel to the longitudinal axis of strips of pressure sensitive adhesive and bonds. Empirically, this was observed to provide similar advantages to the mode of detachment shown in FIG. 6, with an added advantage being a smoother peel (i.e. fluctuations in peel force were greatly attenuated as a function of peel distance). This could provide a significant advantage in user experience, particularly in consumer applications.

### Exemplary Applications

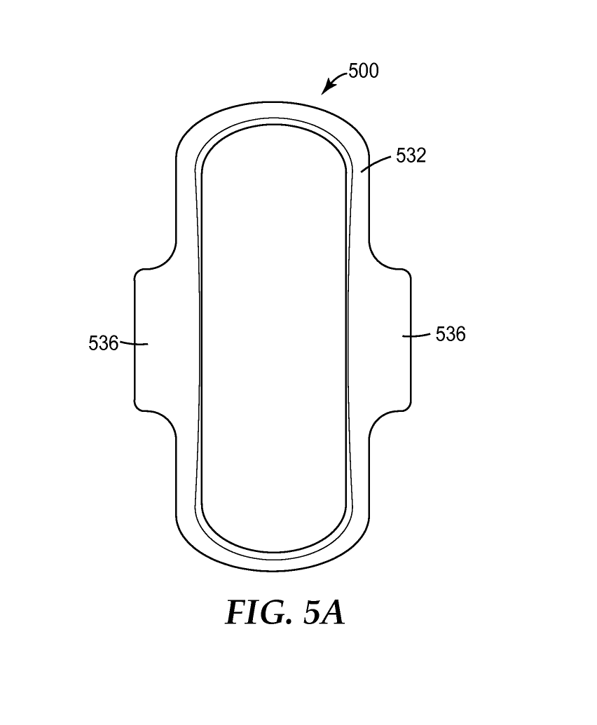

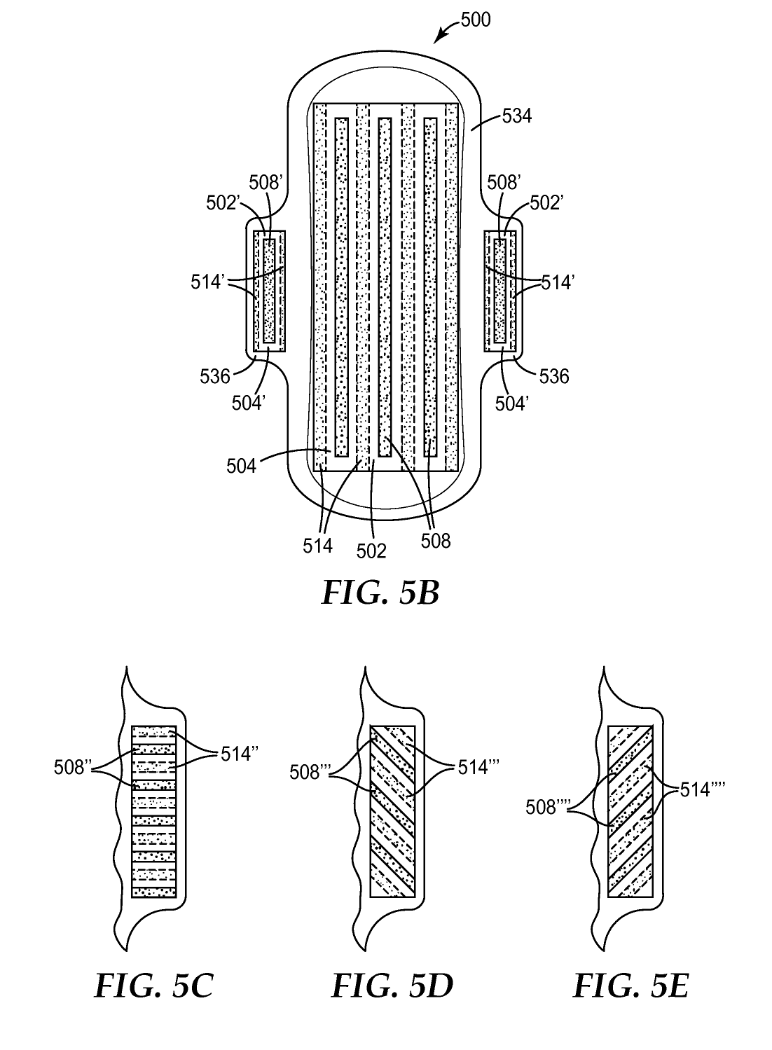

An exemplary stretch release article is illustrated in FIGS. 5A and 5B. The stretch release article 500 is a feminine hygiene pad that attaches to an undergarment by means of pressure sensitive adhesive. The feminine hygiene pad comprises a liquid permeable top sheet 532, a liquid impermeable back sheet 534, and an absorbent core (not shown) therebetween. Typically the top sheet 532 and back sheet 534 extend beyond the absorbent core and are joined to each other, e.g., by gluing or welding by heat or ultrasonic, about the periphery of the absorbent core. However, the top sheet 532 and/or backsheet 534 may also, or alternatively, be joined to the absorbent core. In this particular embodiment, the feminine hygiene pad comprises wings 536 that fold over the lateral edges of the undergarment and adhesively couple to its opposing side for improved attachment to the undergarment.

The liquid permeable topsheet can consist of a nonwoven layer, porous foams, apertured plastic films, etc. Materials suitable for a topsheet are preferably soft and non-irritating to the skin and readily penetrated by fluids.

The liquid impermeable backsheet may consist of a thin plastic film, e.g., a polyethylene or polypropylene film, a nonwoven material coated with a liquid impervious material, a hydrophobic nonwoven material which resists liquid penetration, or laminates of plastic films and nonwoven materials. The backsheet material may be breathable so as to allow vapor to escape from the absorbent core, while still preventing liquids from passing through the backsheet material.

The absorbent core can be made from a variety of conventional absorbent materials. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbents in an absorbent body. It is also common to have absorbent cores comprising layers of different material with different properties with respect to liquid receiving capacity, liquid distribution capacity and storage capacity. Thin absorbent cores often comprise a compressed mixed or layered structure of cellulosic fluff pulp and superabsorbent.

As illustrated in FIGS. 5A and 5B, the stretch release article 500 further comprises an elastic backing 502 having a first side 504 and a second side (not shown) opposite the first side 504. The second side is bonded to the backsheet 534 of the stretch release article 500. A pressure sensitive adhesive 508 is coated on the first side 504 of the elastic backing 502 for subsequent attachment to an undergarment. The patterns of the bonds 514 and pressure sensitive adhesive 508 are similar to those illustrated in FIG. 1. The pressure sensitive 508 is arranged in a first pattern of three parallel strips that extend across the first side 504 of the elastic backing 502 but terminate a short distance from the edges of the elastic backing 502. The bonds 514 are arranged in a second pattern of four parallel strips that extend across the second side of the elastic backing 502. The strips of the first and second patterns are offset so that no overlap occurs between the patterns when projected onto a common reference plane that is coplanar with the elastic backing 502.

A similar construction of elastic backing and pressure sensitive adhesive is applied to each of the wings 536. As illustrated in FIG. 5B, an elastic backing 502′ having a pressure sensitive adhesive 508′ on one side 504′ is bonded through the opposite side to the backsheet 534 of each wing 536. The bonds 514′ and pressure sensitive adhesive 508′ consist of strips that do not overlap when projected onto a common reference plane that is coplanar with the elastic backing 502′. This configuration allows the wings 536 to fold through the leg openings of the undergarment and releasable attach to the underside of the crotch portion of the undergarment.

As mentioned previously, the tactile sensation in peeling a stretch release article from an undergarment depends upon the configuration of bond and pressure sensitive adhesive patterns. FIGS. 5C-5E show alternative patterns comprising strips of bonds 514″, 514′″ and 514″″ and pressure sensitive adhesive 508″, 508′″ and 508″″. The strips can be oriented 90° (FIG. 5C), +45° (FIG. 5D), and −45° (FIG. 5E) relative to the direction of strips in FIG. 5B.

In some embodiments, the stretch release article 500 is packaged with one or more release liners (not shown) to cover and protect the pressure sensitive adhesive 508, 508′. The release liner(s) can be easily peeled away from the stretch release article 500 and discarded prior to use.

The disclosed stretch release articles can provide numerous advantages over conventional means of attachment. First, these configurations can enable use of pressure sensitive adhesives to achieve high bond strength to a substrate, while allowing these pressure sensitive adhesives to cleanly and easily detach from delicate substrates that would otherwise be damaged or destroyed by conventional adhesive constructions. Second, the provided configurations can show surprising resistance to adhesive failure, even when bonded to garments subjected to severe twisting and shearing movements—these types of movements have been observed to cause spontaneous detachment of conventional adhesives. Third, the provided configurations can preserve the above advantages even when removing in peel mode, which is more intuitive to consumers than stretching the adhesive along a direction parallel to the substrate. Fourth, adhesive coating weight can be substantially reduced in the provided configurations compared with conventional adhesives, resulting in reduced manufacturing costs.

### Dual Sides Fasteners

Exemplary dual-sided fasteners are illustrated in FIGS. 8 and 9. The dual-sided fasteners share many of the same elements and features described above with respect to the stretch release articles and patterns in FIGS. 1A-1B, 2A-2C, 3A-3D, 4A-4D and 6. Accordingly, elements and features corresponding to elements and features in the illustrated embodiment of FIGS. 1A-1B, 2A-2C, 3A-3D, 4A-4D and 6 are provided with the same reference numerals in the 800 and 900 series. Reference is made to the description above accompanying FIGS. 1A-1B, 2A-2C, 3A-3D, 4A-4D and 6 for a more complete description of the features and elements (and alternatives to such features and elements) of the embodiments illustrated in FIGS. 8 and 9.

FIG. 8 illustrates a stretch release article 800 that functions as a dual-sided fastener. The fastener 800 comprises an elastic backing 802 having a first side 804 and a second side 806 opposite the first side 804. A pressure sensitive adhesive 808 is located on the first side 804 of the elastic backing 802 and exhibits a first pattern 810. The pressure sensitive adhesive 808 releasably joins the fastener 800 to a substrate (not shown).

A component 812 is joined to the second side 806 of the elastic backing 802 by bonds 814 exhibiting a second pattern 816 that does not substantially overlap with the first pattern 810 when both patterns are projected onto a common reference plane coplanar with the elastic backing 802. The component 812 is preferably a layered material such as a film, nonwoven web, paper and cardboard. An adhesive 875 is located on the side of the component 812 opposite that bonded to the elastic backing 802. The nature of the adhesive 875 is not particularly limiting and may include hot melt adhesives, structural adhesives, and pressure sensitive adhesives. Preferably the adhesive is a pressure sensitive adhesive that may be releasably joined to a second substrate (also not shown). The pressure sensitive adhesive may be of the same type or of a different type than that located on the first side 804 of the elastic backing 802. As illustrated in FIG. 8, the adhesive 875 is a continuous film. However, it should be understood that the adhesive could be continuous or discontinuous and include any type of pattern (e.g., strips, dots, swirls, etc.).

In some embodiments, a release liner may be added to the pressure sensitive adhesive 808 and/or the adhesive 875 to prevent contamination during storage and transport. In some embodiments, the fastener is provided in sheet form. In some embodiments, the fastener is provided as a roll for compact storage and dispensing.

FIG. 9 illustrates and alternative dual-sided fastener 901 in which the end user bonds the elastic backing 902 to the component (not shown). The fastener 901 comprises an elastic backing 902 having a first side 904 and a second side 906 opposite the first side 904. A pressure sensitive adhesive 908 is located on the first side 904 of the elastic backing 902 and exhibits a first pattern 910. The pressure sensitive adhesive 908 releasably joins the fastener 800 to a substrate (not shown). A bonding element 915 is arranged on the second side of the elastic backing 902 in a second pattern 916 that does not substantially overlap with the first pattern 910 when projected onto a common reference plane coplanar with the elastic backing 902. The bonding element 915 is joined to the component (also not shown) by a downstream user. The bonding element 915 is typically not a pressure sensitive adhesive but can be any other bond-forming material as long as it is storable, transportable, and relatively easy for the consumer to use. It is also important that the second pattern 916 of bonding element 915 not change significantly when adhered to the component so that the above described benefits of the stretch release properties may be obtained.

In one embodiment, the bonding element is a hook or loop element of a hook-and-loop mechanical fastener that may be bonded to a component containing loop or hook material, respectively. In another embodiment, the bonding element is a hot melt adhesive that may be reheated upon joining fastener 901 to the component.

In some embodiments, a release liner may be added to pressure sensitive adhesive 908 to prevent contamination during storage and transport. In some embodiments, the fastener is provided in sheet form. In some embodiments, the fastener is provided as a roll for compact storage and dispensing.

### Some Embodiments of the Disclosure

In a first embodiment, the present disclosure provides a stretch release article comprising: an elastic backing having a first side and a second side opposite the first side; a pressure sensitive adhesive on the first side of the elastic backing, the pressure sensitive adhesive exhibiting a first pattern; and a component joined to the second side of the elastic backing by bonds exhibiting a second pattern; where the bonds are not created by a pressure sensitive adhesive, and where the first and second patterns do not substantially overlap with each other when projected in the same plane.

In a second embodiment, the present disclosure provides the article of the first embodiment, wherein the elastic backing comprises a polymeric film.

In a third embodiment, the present disclosure provides the article of the first embodiment, wherein the elastic backing comprises a polymeric nonwoven web.

In a fourth embodiment, the present disclosure provides the article of any one of the first to third embodiments, wherein the elastic backing has an elastic recovery from about 70 percent to about 95 percent based on an elongation of 100 percent.

In a fifth embodiment, the present disclosure provides the article of any one of the first to fourth embodiments, wherein the bonds are formed by at least one of gluing, welding, stapling, sewing and fastening with a hook-and-loop mechanical fastener.

In a sixth embodiment, the present disclosure provides the article of any one of the first to fifth embodiments, further comprising an adhesive on the side of the component opposite the side joined to the elastic backing.

In a seventh embodiment, the present disclosure provides the article of the sixth embodiment, wherein a release liner covers at least one of the pressure sensitive adhesive on the first side of the elastic backing and the adhesive on the side of the component opposite the side joined to the elastic backing.

In an eighth embodiment, the present disclosure provides the article of any one of the first to seventh embodiments, wherein the component is a disposable absorbent article.

In a ninth embodiment, the present disclosure provides the article of the eighth embodiment, wherein the disposable absorbent article is a feminine hygiene pad.

In a tenth embodiment, the present disclosure provides the article of any one of the first to ninth embodiments, wherein the first pattern comprises parallel strips extending in a first direction, and the second pattern comprises parallel strips extending in a second direction.

In an eleventh embodiment, the present disclosure provides the article of the tenth embodiment, wherein the first direction is perpendicular to the second direction.

In a twelfth embodiment, the present disclosure provides the article of the tenth embodiment, wherein the first direction is parallel to the second direction.

In a thirteenth embodiment, the present disclosure provides the article of any one of the tenth to twelfth embodiments, wherein the strips in the first pattern do not extend all the way to the ends of the elastic backing.

In a fourteenth embodiment, the present disclosure provides the article of any one of the tenth, twelfth or thirteenth embodiments, wherein the strips in the first pattern are offset from the strips in the second pattern by a transverse gap.

In a fifteenth embodiment, the present disclosure provides the article of any one of the first to fourteenth embodiments, wherein the first and second patterns each comprise an array of shapes selected from the group consisting of strips, triangles, rectangles, circles and combinations thereof.

In a sixteenth embodiment, the present disclosure provides the article of any one of the first to fourteenth embodiments, wherein at least one of the first and second patterns comprises one or more continuous spirals.

In a seventeenth embodiment, the present disclosure provides the article of any one of the first to sixteenth embodiments, wherein the first and second patterns collectively extend across an area representing about 10 percent to about 60 percent of the overall surface area of the elastic backing.

In an eighteenth embodiment, the present disclosure provides a stretch release fastener comprising: an elastic backing having a first side and a second side opposite the first side; a pressure sensitive adhesive on the first side of the elastic backing, the pressure sensitive adhesive exhibiting a first pattern; a bonding element on the second side of the elastic backing, the bonding element exhibiting a second pattern; where the bonding element is not a pressure sensitive adhesive, and where the first and second patterns do not substantially overlap with each other when projected in the same plane.

In a nineteenth embodiment, the present disclosure provides the fastener of the eighteenth embodiment, wherein the elastic backing comprises a polymeric film.

In a twentieth embodiment, the present disclosure provides the fastener of the eighteenth embodiment, wherein the elastic backing comprises a polymeric nonwoven web.

In a twenty-first embodiment, the present disclosure provides the fastener of any one of the eighteenth to twentieth embodiments, wherein the elastic backing has elastic recovery from about 70 percent to about 95 percent based on an elongation of 100 percent.

In a twenty-second embodiment, the present disclosure provides the fastener of any one of the eighteenth to twenty-first embodiments, wherein the bonding element is at least one of a hot melt adhesive and a heat activated adhesive.

In a twenty-third embodiment, the present disclosure provides the fastener of any one of the eighteenth to twenty-first embodiments, wherein the bonding element is at least one of a hook element and a loop element of a hook-and-loop mechanical fastener.

In a twenty-fourth embodiment, the present disclosure provides the fastener of any one of the eighteenth to twenty-third embodiments, wherein the first pattern comprises parallel strips extending in a first direction, and the second pattern comprises parallel strips extending in a second direction.

In a twenty-fifth embodiment, the present disclosure provides the fastener of the twenty-fourth embodiment, wherein the first direction is perpendicular to the second direction.

In a twenty-sixth embodiment, the present disclosure provides the fastener of the twenty-fourth embodiment, wherein the first direction is parallel to the second direction.

In a twenty-seventh embodiment, the present disclosure provides the fastener of any one of the twenty-fourth to twenty-sixth embodiments, wherein the strips in the first pattern do not extend all the way to the ends of the elastic backing.

In a twenty-eighth embodiment, the present disclosure provides the fastener of any one of the twenty-fourth, twenty-sixth or twenty-seventh embodiments, wherein the strips in the first pattern are offset from the strips in the second pattern by a transverse gap.

In a twenty-ninth embodiment, the present disclosure provides the fastener of any one of the eighteenth to twenty-eighth embodiments, wherein the first and second patterns each comprise an array of shapes selected from the group consisting of strips, triangles, rectangles, circles and combinations thereof.

In a thirtieth embodiment, the present disclosure provides the fastener of any one of the eighteenth to twenty-eighth embodiments, wherein at least one of the first and second patterns comprises one or more continuous spirals.

In a thirty-first embodiment, the present disclosure provides the fastener of any one of the eighteenth to thirtieth embodiments, wherein the first and second patterns collectively extend across an area representing about 10 percent to about 60 percent of the overall surface area of the elastic backing.

### EXAMPLES

The following examples are presented to illustrate some of the advantages of the above stretch release articles and fasteners and are not intended in any way to otherwise limit the scope of the invention.

### Materials

Kraton® D1161 P—a linear styrene-isoprene-styrene copolymer from Kraton Polymers in Houston, Tex. USA.
Escorez™ 1310—an aliphatic hydrocarbon resin from ExxonMobil Chemical in Houston, Tex. USA.
Calsol 5550 Oil—heavy hydrotreated naphthenic distillates from Calumet Lubricants Company in Indianapolis, Ind. USA.
Irganox® 1076—octadecyl-3-(3,5-di-tert butyl-4-hydroxyphenyl)-propionate from BASF in Florham Park, N.J. USA.
B-430 Series Elastic Film—a three-layer laminate consisting of an anisotropic elastic core and a micro-activated, non-elastic polypropylene skin layer surrounding the core from 3M Company in St. Paul, Minn. USA. The B-430 Series Elastic served as the elastic backing of the stretch release articles in the below examples.
PSA Coated Liner—a pressure sensitive adhesive consisting of Kraton® 1161 (48 wt. %), Excorez™ 1310 (49.5 wt. %), 55500 Oil (1.5 wt. %), and Irganox® 1076 (1 wt. %) coated onto one side of a release liner. The ingredients were combined in a solvent mixture of 3 parts toluene/1 part heptane (30% solids), knife coated onto a release liner, and dried in an oven at 65° C. for 20 minutes. The final coating weight was approximately 31 gsm (grams per square meter).
Polypropylene Film—a 6.3 cm (width)×22.5 cm (length) by 180 μm (thick) piece of film having a basis weight of 191 gsm. The Polypropylene Film served as the component of the stretch release articles in the below Examples.
Ad-Tech Multi-Temp Glue Stick—available from Adhesive Technologies Inc. in Hampton, N.H. USA.

### Test Methods

The below test methods employed an Instron Model 55564 HS instrument equipped with a computer for data recording. All sample testing was conducted at constant temperature (23° C.±2° C.) and at constant relative humidity (50%±5%). The instrument crosshead speed was set to 12 inches/min (30.5 cm/min).

All samples were prepared by removing the release liners from the article to expose the pressure sensitive adhesive, applying the pressure sensitive adhesive side of the article to a glass plate (20 cm long by 10 cm wide by 4.5 mm thick), and either passing a 4.5 lb rubber coated roller over the article twice (E-1, E-3, C-1 and C-3) or applying finger pressure to the article (E-2, E-4, C-2 or C-4) to ensure bonding to the glass.

### Peel

One end of the Polypropylene Film was placed in the upper jaw of the Instron instrument, while the glass plate was placed into a 135° stationary jig in the lower jaw of the Instron instrument. The instrument was started and the stress-elongation curve was measured. Average load (g(f)) and area under the curve (energy, mJ) were reported. The peel was performed in a direction aligned with the strips of pressure sensitive adhesive (E-1, E-2, C-1 and C-2) and a direction perpendicular to the strips of pressure sensitive adhesive (E-3, E-4, C-3 and C-4).

### Shear

One end of the Polypropylene Film was placed in the upper jaw of the Instron instrument, while the glass plate was placed into the lower jaw of the Instron instrument. The instrument was started and the stress-elongation curve was measured. Maximum load (g(f)) was determined. The distance the film was pulled until it detached from the glass plate was also determined (mm). The sheer was performed in a direction aligned with the strips of pressure sensitive adhesive (E-1, E-2, C-1 and C-2) and a direction perpendicular to the strips of pressure sensitive adhesive (E-3, E-4, C-3 and C-4).

### Example 1 (E-1)

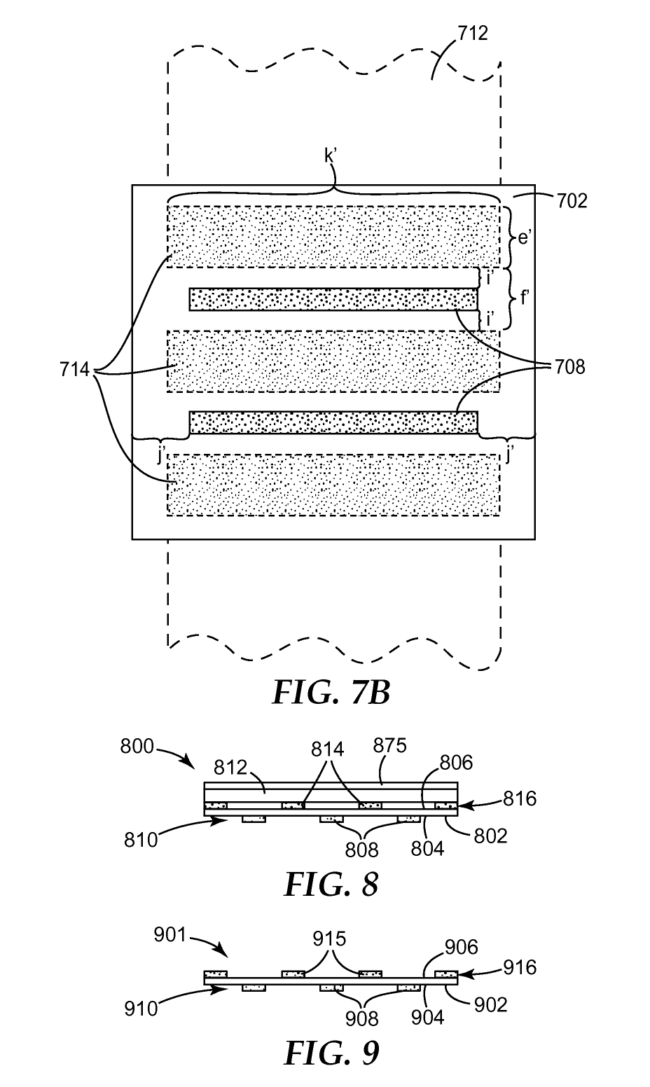

A stretch release article was prepared as follows: A 65 mm (width) by 75 mm (length) piece of B-430 Series Elastic Film 702 (length oriented in MD direction of the Film) was centered on a piece of Polypropylene Film 712, as illustrated in FIG. 7A. The lengths of each Film 702, 712 were aligned in the same direction. A Type 210HC heat sealer from American International Electric Inc. (City of Industry, California, USA) was used to bond the Elastic Film 702 to the Polypropylene Film 712. Three heat seal bonds 714 ran the length of the Elastic Film 702. Each heat seal bond 714 had a width “e” of 1 cm and was separated from adjacent bonds by a distance “f” of 1 cm. Pressure sensitive adhesive 708 was transferred to the side of the Elastic Film 702 facing away from the Polypropylene Film 712 using two strips (5 mm wide by 69 mm long) of PSA Coated Liner. Each piece of PSA Coated Liner was centered between adjacent heat seal bonds 714, as illustrated in FIG. 7A. The longitudinal edge of the PSA Coated Liner was a distance “i” of 2.5 mm from the longitudinal edge of adjacent bonds 714 when projected through the Elastic Film. The PSA Coated Liner did not run the length of the Elastic Film 702 but instead terminated a distance “j” of 3 mm from each edge of the Elastic Film 702.

### Example 2 (E-2)

A stretch release article was prepared as follows: A 65 mm (width) by 75 mm (length) piece of B-430 Series Elastic Film 702 (length oriented in MD direction of the Film) was joined to a piece of Polypropylene Film 712 using a bond pattern similar to that described in Example 1 and Illustrated in FIG. 7A, except that a hot melt adhesive was used in place of the heat sealer. The hot melt adhesive, from an Ad-Tech Multi-Temp Glue Stick, was applied in three strips using a 20 watt Hi Temp Project Pro Glue Gun (mini size) available from Adhesive Technologies Inc. in Hampton, N.H. USA. The three bonds 714 ran the length of the Elastic Film 702. Each bond 714 had a width “e” of 2-4 mm and was separated from adjacent bonds by a distance “f” of 9 mm. Pressure sensitive adhesive 708 was applied to the side of the Elastic Film 702 facing away from the Polypropylene Film 712 using two strips (5 mm wide by 69 mm long) of PSA Coated Liner. Each piece of PSA Coated Liner was centered between adjacent bonds 714, as illustrated in FIG. 7A. The longitudinal edge of the PSA Coated Liner was a distance “i” of 2 mm from the longitudinal edge of adjacent bonds 714 when projected through the Elastic Film. The PSA Coated Liner did not run the length of the Elastic Film 702 but instead terminated a distance “j” of 3 mm from each edge of the Elastic Film 702.

### Example 3 (E-3)

The stretch release article was prepared according to Example 1 except that the Elastic Film 702, heat seal bonds 714 and strips of pressure sensitive adhesive 708 were essentially rotated on the Polypropylene Film 712 by 90°, as illustrated in FIG. 7B. The heat seal bonds 714 had a width “e′” of 1 cm and a length “k′” of 63 cm. The heat seal bonds 714 were separated from adjacent bonds by a distance “f′” of 1 cm. The two strips of PSA Coated Liner where 5 mm wide by 58 mm long and each was centered between adjacent heat seal bonds 714. The longitudinal edge of the PSA Coated Liner was a distance “i′” of 2.5 mm from the longitudinal edge of adjacent bonds 714 when projected through the Elastic Film. The PSA Coated Tape did not run the length of the Elastic Film 702 but instead terminated a distance “j′” of 8.5 mm from each edge of the Elastic Film 702.

### Example 4 (E-4)

The stretch release article was prepared according to Example 2 except that the Elastic Film 702, adhesive bonds 714 and strips of pressure sensitive adhesive 708 were essentially rotated on the Polypropylene Film 712 by 90°, as illustrated in FIG. 7B. The adhesive bonds 714 had a width “e′” of 2-4 mm and a length “k′” of 63 cm. The adhesive bonds 714 were separated from adjacent bonds by a distance “f′” of 9 mm. The two strips of PSA Coated Liner where 5 mm wide by 58 mm long and each was centered between adjacent bonds 714. The longitudinal edge of the PSA Coated Liner was a distance “i′” of 2 mm from the longitudinal edge of adjacent bonds 714 when projected through the Elastic Film. The PSA Coated Liner did not run the length of the Elastic Film 702 but instead terminated a distance “j′” of 8.5 mm from each edge of the Elastic Film 702.

### Comparative Example 1 (C-1)

An article was prepared as follows: A piece of PSA Coated Liner (55 mm wide and 75 mm long) was centered on a piece of the Polypropylene Film in both the length and width directions, with the lengths of the Liner and Film aligned in the same direction. The release liner was removed to expose the pressure sensitive adhesive. A piece of the B-430 Series Elastic Film (65 mm wide and 75 mm long) was centered on the Polypropylene Film in both the length and width directions with the lengths of the Films extending in the same direction. Two strips of PSA Coated Liner (5 mm wide by 69 mm long) were applied to the side of the Elastic Film facing away from the Polypropylene Film. The two strips were centered on the Elastic Film in both the length and width directions and separated by 1 cm in the width direction of the Elastic Film. The PSA Coated Liners did not run the entire length of the Elastic Film but instead terminated 3 mm from each edge of the Elastic Film.

### Comparative Example 2 (C-2)

An article was prepared as follows: Two strips (5 mm wide by 69 mm long) of PSA Coated Liner were applied to a piece of Polypropylene Film such that the strips were aligned with the length direction of the Film. The two strips were centered on the Polypropylene Film in both the length and width directions and separated by 1 cm in the width direction of the Film.

### Comparative Example 3 (C-3)

An article was prepared as follows: A piece of PSA Coated Liner (55 mm wide and 63 mm long) was centered on a piece of the Polypropylene Film, with the lengths of the Liner and Polypropylene Film aligned in the same direction. The release liner was removed to expose the pressure sensitive adhesive. A piece of the B-430 Series Elastic Film (65 mm wide and 75 mm long) was centered on the Polypropylene Film in both the length and width direction with the length of the Elastic Film extending across the width of the Polypropylene Film. Two strips of PSA Coated Liner (5 mm wide by 58 mm long) were applied to the side of the Elastic Film facing away from the Polypropylene Film. The two strips were centered on the Elastic Film in both the length and width dimensions and separated by 1 cm in the width direction of the Elastic Film. The PSA Coated Liner did not run the entire length of the Elastic Film but instead terminated 8.5 mm from each edge of the Elastic Film.

### Comparative Example 4 (C-4)

An article was prepared as follows: Two strips (5 mm wide by 58 mm long) of PSA Coated Liner were applied to a piece of Polypropylene Film such that the lengths of the strips were oriented with the width of the Polypropylene Film. The two strips of PSA Coated Liner were centered on the Polypropylene Film in both length and wide directions and separated by 1 cm in the length direction of the Polypropylene Film.

### Results

The results from the peel and sheer testing are provided in Tables 1 and 2 below.

TABLE 1
Sheer Testing^{a}

| Sample | Max. Load (gf) | Extension to Max Load (mm) |
|---|---|---|
| E-1 | 9719 | 37 |
| E-2 | 10616 | 22 |
| C-1 | 29600 | 5 |
| C-2 | 28868 | 5 |
| E-3 | 7847 | 25 |
| E-4 | 9562 | 19 |
| C-3 | 31184 | 5 |
| C-4 | 31151 | 5 |

^{a}Values are the average of two replicates

TABLE 2
Peel Adhesion

| | Max. Load | Ave. Load | Ave. Peak | Energy | Amount of Residual Adhesive Left |
|---|---|---|---|---|---|
| Sample | (gf) | (gf) | (gf) | (mJ) | on Glass Substrate |
| E-1 | 488^{a} | 332^{a} | 380^{a} | 384^{a} | No residual adhesive for two of the |
| | | | | | three samples. A tiny speck of |
| | | | | | residual adhesive remained for the |
| | | | | | third sample. |
| E-2 | 514^{a} | 340^{a} | 421^{a} | 405^{a} | No residual adhesive. |
| C-1 | 753^{a} | 394^{a} | 599^{a} | 523^{a} | Greater than 50% residual |
| | | | | | adhesive. |
| C-2 | 613^{a} | 374^{a} | 494^{a} | 411^{a} | No residual adhesive. However, |
| | | | | | the adhesive delaminated from the |
| | | | | | elastic backing. |
| E-3 | 1280^{b} | 527^{b} | 1073^{b} | 424^{b} | No residual adhesive. |
| E-4 | 1353^{b} | 670^{c} | 1113^{b} | 399^{c} | No residual adhesive. |
| C-3 | 3944^{b} | 878^{b} | 3219^{b} | 527^{b} | Greater than 50% residual |
| | | | | | adhesive. |
| | | | | | Also, the adhesive delaminated |
| | | | | | from the elastic backing. |
| C-4 | 3166^{b} | 646^{b} | 2654^{b} | 384^{b} | No residual adhesive. However, |
| | | | | | the adhesive partially delaminated |
| | | | | | from the elastic backing. |

^{a}Value based on average of three replicates.
^{b}Value based on average of two replicates.
^{c}Value based on a single measurement.

The embodiments described above and illustrated in the figures are presented by way of example only and are not intended as a limitation upon the concepts and principles of the present invention.

Thus, the invention provides, among other things, stretch release articles and fasteners. Various features and advantages of the invention are set forth in the following claims.

1. A stretch release article comprising:
an elastic backing having a first side and a second side opposite the first side;
a pressure sensitive adhesive on the first side of the elastic backing, the pressure sensitive adhesive exhibiting a first pattern; and
a component joined to the second side of the elastic backing by bonds exhibiting a second pattern;
where the bonds are not created by a pressure sensitive adhesive, and
where the first and second patterns do not substantially overlap with each other when projected in the same plane.

2. The article of claim 1, wherein the elastic backing comprises a polymeric film.

3. The article of claim 1, wherein the elastic backing comprises a polymeric nonwoven web.

4. The article of claim 1, wherein the elastic backing has an elastic recovery from about 70 percent to about 95 percent based on an elongation of 100 percent.

5. The article of claim 1, wherein the bonds are formed by at least one of gluing, welding, stapling, sewing and fastening with a hook-and-loop mechanical fastener.

6. The article of claim 1, further comprising an adhesive on the side of the component opposite the side joined to the elastic backing.

7. The article of claim 6, wherein a release liner covers at least one of the pressure sensitive adhesive on the first side of the elastic backing and the adhesive on the side of the component opposite the side joined to the elastic backing.

8. The article of claim 1, wherein the component is a disposable absorbent article.

9. The article of claim 8, wherein the disposable absorbent article is a feminine hygiene pad.

10. The article of claim 1, wherein the first pattern comprises parallel strips extending in a first direction, and the second pattern comprises parallel strips extending in a second direction.

11. The article of claim 10, wherein the first direction is perpendicular to the second direction.

12. The article of claim 10, wherein the first direction is parallel to the second direction.

13. The article of claim 12, wherein the strips in the first pattern do not extend all the way to the ends of the elastic backing.

14. The article of claim 12, wherein the strips in the first pattern are offset from the strips in the second pattern by a transverse gap.

15. The article of claim 1, wherein the first and second patterns each comprise an array of shapes selected from the group consisting of strips, triangles, rectangles, circles and combinations thereof.

16. The article of claim 1, wherein at least one of the first and second patterns comprises one or more continuous spirals.

17. The article of claim 1, wherein the first and second patterns collectively extend across an area representing about 10 percent to about 60 percent of the overall surface area of the elastic backing.

18. A stretch release fastener comprising:
an elastic backing having a first side and a second side opposite the first side;
a pressure sensitive adhesive on the first side of the elastic backing, the pressure sensitive adhesive exhibiting a first pattern;
a bonding element on the second side of the elastic backing, the bonding element exhibiting a second pattern;
where the bonding element is not a pressure sensitive adhesive, and
where the first and second patterns do not substantially overlap with each other when projected in the same plane.

19. The fastener of claim 18, wherein the elastic backing comprises a polymeric film.

20. The fastener of claim 18, wherein the elastic backing comprises a polymeric nonwoven web.

21. The fastener of claim 18, wherein the elastic backing has elastic recovery from about 70 percent to about 95 percent based on an elongation of 100 percent.

22. The fastener of claim 18, wherein the bonding element is at least one of a hot melt adhesive and a heat activated adhesive.

23. The fastener of claim 18, wherein the bonding element is at least one of a hook element and a loop element of a hook-and-loop mechanical fastener.

24. The fastener of claim 18, wherein the first pattern comprises parallel strips extending in a first direction, and the second pattern comprises parallel strips extending in a second direction.

25. The fastener of claim 24, wherein the first direction is perpendicular to the second direction.

26. The fastener of claim 24, wherein the first direction is parallel to the second direction.

27. The fastener of claim 26, wherein the strips in the first pattern do not extend all the way to the ends of the elastic backing.

28. The fastener of claim 26, wherein the strips in the first pattern are offset from the strips in the second pattern by a transverse gap.

29. The article of claim 18, wherein the first and second patterns each comprise an array of shapes selected from the group consisting of strips, triangles, rectangles, circles and combinations thereof.

30. The article of claim 18, wherein at least one of the first and second patterns comprises one or more continuous spirals.

31. The article of claim 18, wherein the first and second patterns collectively extend across an area representing about 10 percent to about 60 percent of the overall surface area of the elastic backing.
